(19) United States
(12) Patent Application Publication
(10) Pub. No.: US 2006/0097863 A1
(43) Pub. Date: May 11, 2006

(54) Tracking equipment

(75) Inventors: Eric J. Horowitz, Pikesville, MD (US); Scott J. Horowitz, Park City, UT (US); Seymour Bernard Horowitz, Thousand Oaks, CA (US)

Correspondence Address: DAVID LEWIS, 1250 AVIATION AVE., SUITE 200B, SAN JOSE, CA, 95110, US

(73) Assignee: Eric Horowitz

(21) Appl. No.: 11/077,350

(22) Filed: Mar. 10, 2005

Related U.S. Application Data
(60) Provisional application No. 60/620,773, filed on Oct. 21, 2004.

(51) Int. Cl. G08B 19/00; G06F 17/30; C12Q 1/68; G08B 1/00; G08B 21/00

(52) U.S. Cl. 340/521; 707/10; 250/200; 340/531; 340/679

(57) ABSTRACT

In an embodiment, a first set of modules is placed on pieces of equipment for providing information about the piece of equipment on which the modules are located. In another embodiment, a second set of modules is placed on racks where pieces of equipment may be stored. In yet another embodiment, modules of the second set communicate with modules of the first set in order to provide information about the equipment and where the equipment is stored. In an embodiment, the modules on the racks also provide information about the availability of space on the racks. In an embodiment, modules of the second set provide power to modules of the first set. In yet another embodiment, the first set and second set of modules communicate with each other optically without being physically connected.

### CROSS-REFERENCE TO RELATED APPLICATIONS

This application claims priority benefit of U.S. Provisional Patent Application No. 60/620,773, filed Oct. 21, 2004, which is incorporated herein by reference.

### FIELD OF THE INVENITON

The disclosure is related to keeping track of items of interest, such as pieces of equipment.

### BACKGROUND OF THE INVENTION

Keeping track of items of interest that reside in large storage areas in at least certain situations is desirable. A record of the locations of items of interest may be maintained manually. Another possible method of maintaining the locations of items of interest is by attaching Radio Frequency Identification (RFID) tags to the items of interest. However, Radio Frequency Identification (RFID) tags are not always convenient, because RFIDs may not provide a precise enough location to determine a specific rack or slot in which the items of interest are located.

### BREIF DESCRIPTION OF THE FIGURES

In the following drawings like reference numbers are used to refer to like elements. Although the following figures depict various examples of the invention, the invention is not limited to the examples depicted in the figures.

FIG. 1 shows an example of a system that may be useful tracking an item of interest.

FIG. 2A shows one example of a rack that may be used in the system of FIG. 1.

FIG. 2B shows another example of a rack that may be used in the system of FIG. 1.

FIG. 3A shows an example of a portion of the wall of the rack of FIG. 2A and an example of a piece of equipment at a location associated with the portion.

FIG. 3B shows an example of a security tape and the manner in which the security tape is mounted within the system of FIG. 3A.

FIG. 3C shows an example of a circuit diagram of an Equipment Information Module(EIM) of FIG. 3A.

FIG. 3D shows an example of a circuit diagram of associated with the Equipment Discovery Module (EDM) of FIG. 3A.

FIG. 3E shows an example of a circuit diagram of an Equipment Discovery Module (EDM) of FIG. 3A.

FIG. 4A shows an example of a block diagram of a console used in the system of FIG. 1.

FIG. 4B shows an example of a memory system associated with the console of FIG. 4A.

FIG. 5 shows an example of a screenshot from a Graphical User Instrument (GUI), which may be associated with the user interface of FIG. 4B and the system of FIG. 1.

FIG. 6A shows another example of a screenshot associated with the system of FIG. 1.

FIG. 6B shows an example of an information window that displays information about a selected piece of equipment of FIG. 6A.

FIG. 6C shows an example of an information window that displays information about a selected piece of equipment of FIG. 6A in which the piece of equipment was not identifiable.

FIG. 6D shows an example of an information window for a rack.

FIG. 6E shows an example of an information window for a row.

FIG. 6F shows an example of an information window for a position.

FIG. 6G shows an example of a search window associated with the system of FIG. 1.

FIG. 6H shows an example of a log for monitoring changes in the system of FIG. 1 as the changes occur.

FIG. 7 shows an example of a method of converting a system into one, such as the system FIG. 1, which tracks items of interest, such as pieces of equipment.

FIG. 8 shows an example of a manner in which the system FIG. 1 may operate.

### DETAILED DESCRIPTION OF SOME EXAMPLES OF THE INVENTION

Although various embodiments of the invention may have been motivated by various deficiencies with the prior art, which may be discussed or alluded to in one or more places in the specification, the embodiments of the invention do not necessarily address any of these deficiencies. In other words, different embodiments of the invention may address different deficiencies that may be discussed in the specification. Some embodiments may only partially address some deficiencies that may be discussed in the specification, and some embodiments may not address any of these deficiencies.

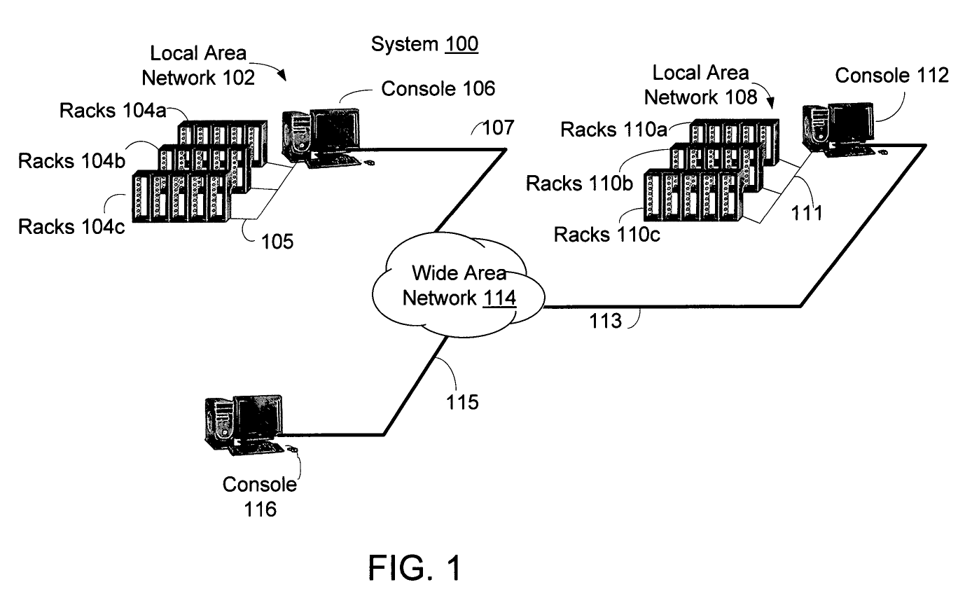

In general, at the beginning of the discussion of each of FIGS. 1-6H is a brief description of each element. The brief discussion may consist of no more than listing the name of each of the elements in the one of FIGS. 1-6H that is being discussed. After the brief description of each element, each element is further discussed in numerical order. In general, each of FIGS. 1-8 is discussed in numerical order and the elements within FIGS. 1-8 are also usually discussed in numerical order to facilitate easily locating the discussion of a particular element. Nonetheless, there is no one location where all of the information of any element of FIGS. 1-8 is necessarily located. Unique information about any particular element or any other aspect of any of FIGS. 1-8 may be found in, or implied by, any part of the specification.

In various places in discussing the drawings a range of letters, such as “a-c,” “a-e,” “a-g,” “a-j,” “a-n,” “a-r,” or “a-s” are used to refer to individual elements of various series of elements that are the same. In each of these series, the ending letters are integer variables that can be any number. Unless indicated otherwise, the number of elements in each of these series are unrelated to one another. Specifically, even though one letter (e.g. “c”) comes earlier in the alphabet than another letter (e.g., “s”) does not mean that the earlier letter represents a smaller number. The value of the earlier letter is unrelated to the later letter, and may represent a value that is greater than, the same as, or less than the later letter.

FIG. 1 shows system 100, which is an example of a system that tracks items of interest. System 100 may keep an inventory of items of interest. System 100 includes local facility 102. Local facility 102 includes rows of racks 104a-c, connection 105, console 106, and connection 107. System 100 also includes local facility 108, which includes rows of racks 110a-c, connection 111, and console 112 and connection 113. Additionally, system 100 includes wide area network 114, connection 115, and console 116. In other embodiments, system 100 may include additional components and/or may not include all of the components listed above.

System 100 may track pieces of equipment stored within its racks. Additionally system 100 may collect and/or store information about each piece of equipment, such as the precise location of the piece of equipment, characteristics of the piece of equipment, and other information, in addition to, or instead of, the presence or absence of the piece of equipment within the racks. System 100 is discussed further, below, in the course of discussion each of the components of system 100. Unless indicted otherwise, throughout this specification the terms “item(s) of interest,” “piece(s) of equipment,” and “device(s)” may be substituted one for another wherever any of these terms appear to thereby obtain another embodiment.

Local facility 102 and local facility 108 include one or more racks (within which equipment are stored) and a system for tracking the equipment stored in the racks. Each local facility may include or be associated with one or more local area networks. In an embodiment, the location of each piece of equipment within local facilities 102 and 108 may be tracked by system 100.

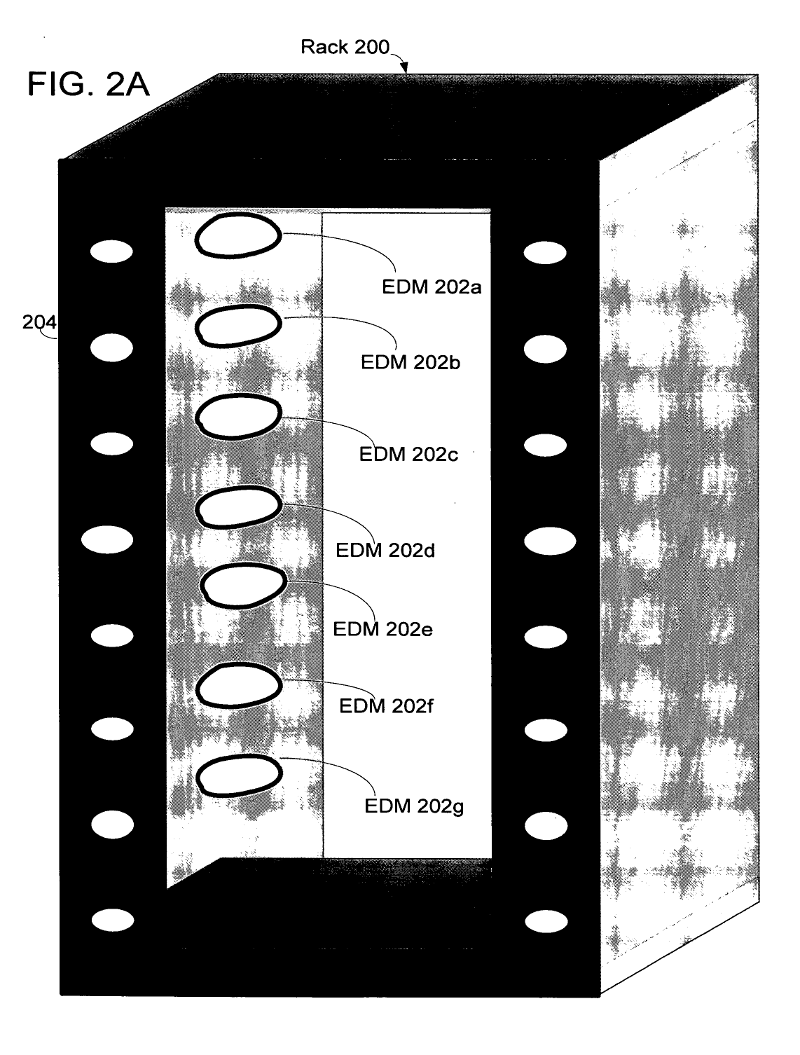

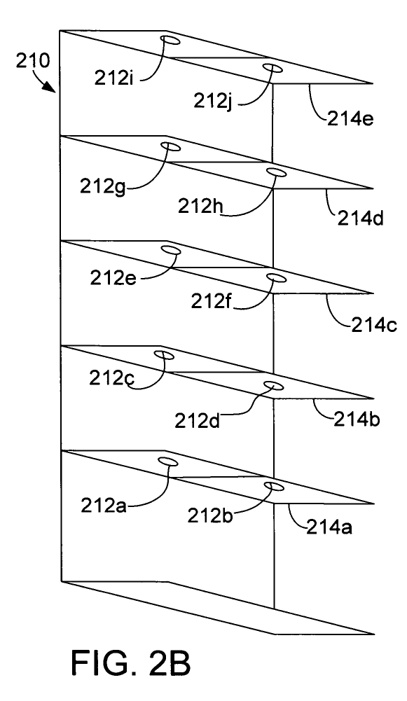

Rows of racks 104a-c and 110a-c store equipment. Rows of racks 104a-c and 110a-c include the racks of local facilities 102 and 108, respectively. The racks within rows of racks 104a-c and 110a-c participate in tracking the equipment located within them. In an embodiment, rows of racks 104a-c and 110a-c send signals to a central location regarding the absence, presence, and location of equipment that is stored or that was removed from rows of racks 104a-c and 110a-c, respectively. Although, as depicted in FIG. 1, each row within rows of racks 104a-c and 110a-c includes only five racks, each row may have any number of racks. Although in FIG. 1, all rows are depicted as having the same number of racks, each row may have a different number of racks than one or more other rows of racks. The number of racks in each row may be chosen independently of one another. Although in the example of FIG. 1 racks are used for storing equipment, in other embodiments other forms of storing equipment could be used, such as bins, shelves, cubby holes, or other receptacles. Although rows of racks 104a-c and 110a-c are depicted as standing upright and side to side with one another, rows of racks 104a-c and 110a-c could be replaced with the racks that lie sideways on the ground, and that are stacked one on top of another. In another embodiment, system 100 may use a mixture of different types of racks. Although FIG. 1 only shows three rows of racks within each of local facilities 102 and 108, each local area network may have any number of rows of racks. Rows of racks 104a-c and 110a-c are discussed further, in conjunction with the discussion of FIGS. 2A and B, below.

Consoles 106, 112, and 116 provide user interfaces for system 100. Consoles 106, 112, and 116 provide information to a user regarding the location of equipment within each of rows of racks 104a-c and 110a-c. Consoles 106, 112, and 116 receive information from rows of racks 104a-c and 110a-c regarding the equipment present in rows of racks 104a-c and 110a-c. In an embodiment, details regarding the characteristics of the pieces of equipment (e.g., the functions, features, make, model, version, and software stored therein) are included within the information sent from rows of racks 104a-c and 110a-c to consoles 106, 112, and 116. Consoles 106, 112, and 116 may be associated with one or more databases or other forms of storing and/or organizing the information sent from rows of racks 104a-c and 110a-c.

In an embodiment, each of consoles 106 and 112 may store the information from its local facilities 102 and 108, respectively. In an embodiment, console 116 can access and/or store information about equipment in any of the local area networks of system 100. In an embodiment, console 116 can access and/or store information about only a selected one of, or only a selected combination of, local area networks of system 100. In an embodiment, only one console of consoles 106, 112, and 116 is capable of accessing information about equipment from any local area network within system 100. In an embodiment, each of consoles 106, 112, and 116 can access information about any local area network of system 100. In an embodiment, each of, any combination of, or all of, consoles 106, 112, and 116 may be able to store and/or access only a part of one or more of local facilities 102 and/or 108 in addition to, or instead of, being able to access all of the information and/or store all of the information about one or more others of the local area networks. Although in FIG. 1 each local area network has only one console, in an embodiment, each of local facilities 102 and 108 may include any number of consoles.

Connections 105 and 111 connect rows of racks 104a-c and 110a-c to consoles 106 and 112, respectively. Similarly, connections 107, 113, and 115 connect consoles 106, 114, and 116 to a wide area network, respectively. In an embodiment, connections 107, 113, and 115 allow consoles 106, 112, and 116 to communicate with, and/or access information from, any of the local area networks of system 100. Each of connections 105, 107, 113, and 115 could be anyone of, or any combination of, one or more copper wire connections, one or more wireless connections, one or more optical connection, and/or any other type of connection capable of communicating information.

Wide area network 114 connects local facilities 102 and 108 and console 116, allowing local facilities 102 and 108 and console 116 to communicate with one another. One example of a wide area network 114 is the Internet.

Although in the example of FIG. 1, system 100 is depicted as including two local facilities 102 and 108, system 100 may have any number of local area networks. Although in the example depicted in FIG. 1 system 100 includes a console 116 linked to the local area networks, console 116 is optional. Similarly, system 100 may include any number of consoles at locations remote from local facilities 102 and 108 in addition to console 116. Additionally, although in FIG. 1 local facility 102, local facility 108, and console 116 are linked via wide area network 114, the networks and consoles of system 100 may be linked in other ways instead. For example, system 100 may include (1) two or more local area networks linked to one another via dedicated links or (2) consoles linked to system 100 via dedicated links. The local area networks and consoles of system 100 may be linked to one another by other local area networks in addition to or instead of dedicated links and/or one more wide area networks. Thus, for example, system 100 may include links between local area networks and/or consoles that are any combination of, or any one of, one or more dedicated links, one or more wide area networks, and/or one or more local area networks.

FIG. 2A shows rack 200, which is one example of a rack that may be used in system 100. Rack 200 includes Equipment Discovery Modules (EDMs) 202a-g located on wall 204. In other embodiments, rack 200 may include additional components and/or may not include all of the components listed above. Rack 200 may be built to meet a variety of standards, such as those of the American National Standards Institute and/or Electronic Industries Association (ANSI/EIA). For example, rack 200 may be built in conformance with ANSI/EIA-310-D-1992.

Each of EDMs 202a-g is located at a position (e.g., a slot) where a piece of equipment (or a unit) can be installed. Each position may include engagement structures such as brackets, pegs, holes, and/or other means for engaging the piece of equipment, and holding the piece of equipment in place. Each of EDMs 202a-g detects whether a piece of equipment is located at the position associated with that EDM. If a piece of equipment is located at a position associated with one of EDMs 202a-g, the EDM may be capable of detecting whether the piece of equipment is improperly installed and/or is a non-compliant piece of equipment. In an embodiment, EDMs 202a-g may be capable of distinguishing between whether a piece of equipment is not present or non-compliant. In an embodiment, if a piece of equipment is located at a position associated with one of EDMs 202a-g, the EDM associated with that location sends information about the equipment to any one of, any combination of, or all of consoles 106, 112, and 116.

In an embodiment, wall 204 supports equipment that is placed in rack 200. EDMs 202a-g are mounted to or embedded within wall 204. Wall 204 includes engagement structures for mounting the pieces of equipment, which may define the positions (e.g., slots) where the pieces of equipment are mounted. EDMs 202a-g are positioned on wall 204 so as to be communicatively coupled (e.g., optically) with corresponding modules on pieces of equipment. EDMs 202a-g may be positioned on wall 204 so as to be close enough to corresponding module on pieces of equipment so that EDMs 202a-g may be easily communicatively coupled (e.g., via a cable) to the corresponding modules. In an embodiment, EDMs 202a-g are located in any of several other locations, such as the back wall of rack 200. In an embodiment there is one EDM at each position associated with wall 204. In other embodiments there may be any number of EDMs at each position of rack 200 for communicating with different modules on the same piece of equipment allowing the modules to be located in a variety of positions on the piece of equipment.

FIG. 2B shows rack 210, which is another example of a rack that may be used in system 100. Rack 210 includes EDMs 212a-j located on shelves 214a-e. In other embodiments, rack 210 may include additional components and/or may not include all of the components listed above.

Similar to rack 200, racks 210 may built to meet the ANSI/EIA-310-D-1992 standard and/or other standards. Similar to rack 200, in rack 210 each of EDMs 212a-j is located at a position where a piece of equipment (or a unit) can be installed. In contrast to rack 200, in rack 210 each of EDMs 212a-j are located on the underside of a shelf. Each position may include brackets, pegs, holes, and/or other means for engaging the piece of equipment, and holding the piece of equipment in place. Each of EDMs 212a-j may be capable of performing all of the functions that each of EDMs 202a-g is capable of. If a piece of equipment is located at a position associated with one of EDMs 212a-j, the EDM associated with that location sends information about the equipment to any one of, any combination of, or all of consoles 106, 112, and 116.

Shelves 214a-e support pieces of equipment. On the underside of shelves 214a-e are located EDMs 212a-j. Each of shelves 214a-e may include engagement structures for holding pieces of equipment in fixed positions. The engagement structures may define positions on shelves 214a-e where pieces of equipment may be located. Similar to EDMs 202a-g, EDMs 212a-j are positioned on shelves 214a-e so as to be communicatively coupled (e.g., optically) with corresponding modules on pieces of equipment. Alternatively, EDMs 212a-j are positioned on shelves 214a-e so as to be close enough to corresponding modules on pieces of equipment so that EDMs 212a-j may be easily communicatively coupled (e.g., via a cable) to the corresponding modules. In an embodiment there is one EDM at each position associated with shelves 214a-e. In other embodiments there may be any number of EDMs at each position on shelves 214a-e for communicating with different modules on the same piece of equipment and/or so that the modules may be located in a variety of positions on the piece of equipment. Additionally, even if there is only one EDM at each position, EDMs 212a-j may be located in a variety of other locations other than the underside of shelves 214a-e, such as the top sides of shelves 214a-e and/or the walls of rack 210. In an alternative embodiment, instead of or in addition to EDMs 202a-g or EDMs 212a-j, rack 200 or rack 210, may include a variety of sensors for detecting the presence of a piece of equipment and/or determining the characteristics of the piece of equipment. For example, racks 200 or 210 may include cameras and software for determining the characteristics of the piece of equipment based on the look of the piece of equipment, the control panel, and/or a variety of markings on the piece of equipment, such as the make and model number. The software may also be capable of optical character recognition for reading the markings on the piece of equipment. If cameras are used, each of the cameras may be positioned for viewing only one piece of equipment, multiple pieces of equipment, all of the pieces of equipment in a rack, all of the pieces of equipment in multiple racks, all of the pieces of equipment in a row, or all of the pieces of equipment in multiple rows, for example.

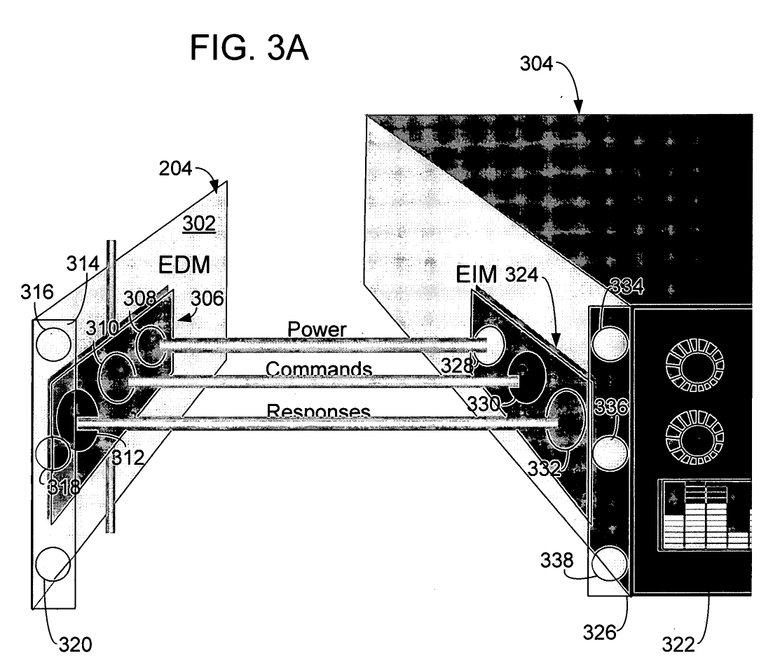

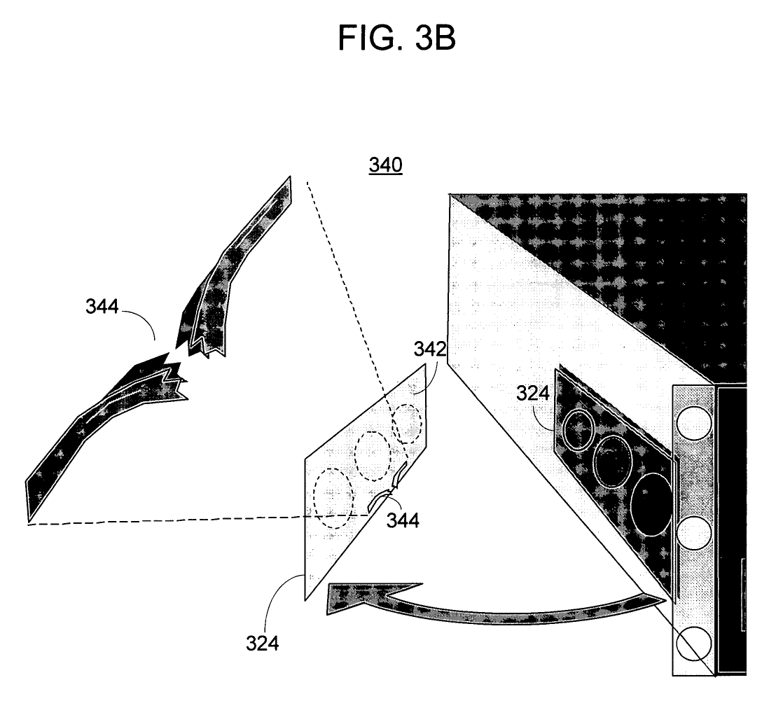

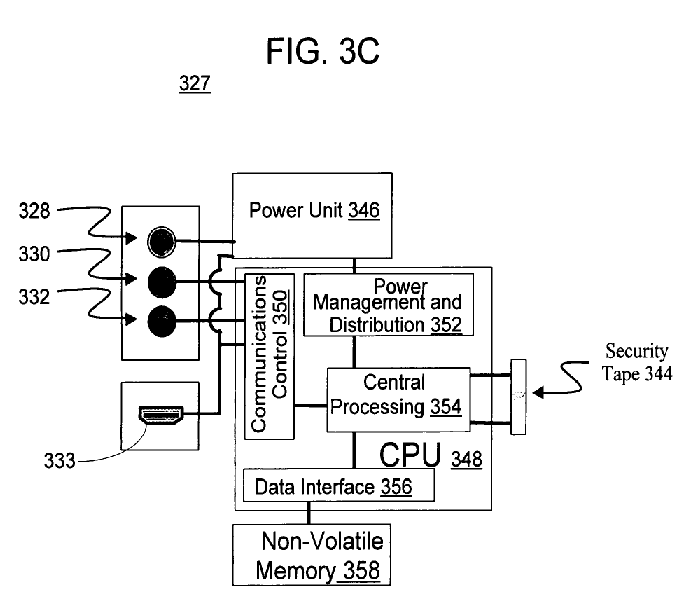

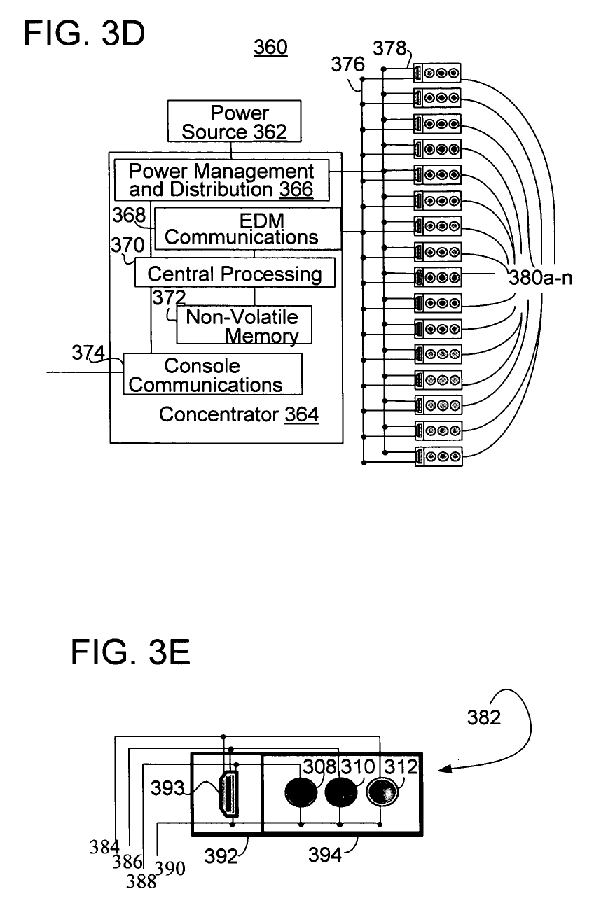

FIG. 3A shows portion 302 of wall 204, which is a wall of rack 200 of FIG. 2A. FIG. 3A also shows a piece of equipment 304 at the location associated with portion 302. Portion 302 includes EDM 306, which has energy source 308, communications transmitter 310, and communications receiver 312. Portion 302 also includes flange 314, which has engagement structures 316, 318, and 320. Piece of equipment 304 includes control panel 322, Equipment Identification Module (EIM) 324, and flange 326. EIM 324 includes energy receiver 328, communications receiver 330, and communications transmitter 332. Flange 326 includes engagement structure 334, 336, and 338. In other embodiments, portion 302 and/or piece of equipment 304 may include additional components and/or may not include all of the components listed above.

Portion 302 may be any of the portions associated with rack 200 (FIG. 2A). Alternatively, portion 302 may be associated with a different type of rack. Portion 302 is just one example of a manner in which a piece of equipment can be attached to system 100.

Piece of equipment 304 is just one example of a piece of equipment that may be attached to system 100. Piece of equipment 304 may be essentially any type of equipment. For example, piece of equipment 304 may be a computer, a phase lock control, a device for controlling a mechanical device, a power supply, a diskdrive, a server, a router, a switch, a bridge, network memory device, a volatile memory device, and/or a non volatile memory device. Each piece of equipment in system 100 may be different from, or the same as, any of, any combination of, or all of the other pieces of equipment of system 100. The separation between the rack 200 and piece of equipment 304 is for illustration, but in reality, the two components will be joined together.

The pieces of equipment within system 100 may cooperate with one another and perform any of a number of operations while installed in system 100. For example, the equipment within system 100 may cooperate together to form a computer, a database, or a network controller, for example. The equipment within system 100 may be organized into any number of subsystems, which may be independent of one another and/or may perform operations that are unrelated to one another. Alternatively, some or all of the subsystems may cooperate with one another to perform any number of operations. Alternatively, any one of, any combination of, or all of the pieces of equipment may be placed in system 100 for storage, and may not perform any function while in system 100 and may not even be functional.

EDM 306 is one example of an EDM, which may be any of EDMs 202a-g. Energy source 308 sends energy to an EIM, and may be used to supply power to the EIM. EDM 306 may provide to a console (e.g., one or more of consoles 106, 112, and/or 116) information identifying the location of piece of equipment 304. The identifying information may include information identifying the local area network, the rack, and/or the position within the rack that is associated with piece of equipment 304. EDM 306 may be capable of detecting whether or not piece of equipment 304 is present or compliant. In an embodiment, EDM 306 may be capable of distinguishing between a missing piece of equipment and a noncompliant piece of equipment.

Energy source 308 may supply power to a module on piece of equipment 304. Energy source 308 may be any of a number of devices (relying on any number of methods) of supplying power. Energy source 308 may connect directly to a module on piece of equipment 304. Alternatively, energy source 308 may send power to the module without being physically connected to the module on piece of equipment 304. For example, energy source 308 may be a light source such as a Light Emitting Diode (LED), an incandescent light bulb, and/or a fluorescent light bulb. Energy source 308 may transmit other forms, and/or wavelengths, of electromagnetic energy, such as microwaves, radio waves, or X-rays, for example. Some other examples of wavelengths of light that may be transmitted by energy source 308 are ultraviolet light and infrared light. Energy source 308 may transmit monochromatic light, a combination of some wavelengths of light, or white light, for example. Energy source 308 may be the end of an optical fiber, which transmits light from a remote light source. Energy source 308 may be a source of electrical energy, such as a battery, an outlet, or a power supply. Energy source 308 may be a source of mechanical energy. Energy source 308 may include any combination of any of the various energy sources listed above and/or any other energy source. Energy source 308 may be a single energy source or may be a system including a combination of any number of energy sources. Energy source 308 may include several energy transmitters each compatible with a different energy receiver. 49 Communications transmitter 310 transmits communications to a module associated with (and/or located at) piece of equipment 304. Communications transmitter 310 may query a module associated with piece of equipment 304 for information about piece of equipment 304. Communications transmitter 310 may only transmit a single type of signal, which represents a statement that requests all of the information available about piece of equipment 304. Alternatively, communications transmitter 310 may be capable of sending signals chosen from a set of many different signals in which each signal may represent a different statement or may represent a portion of a statement. Each statement may cause a different operation to be performed by a module associated with (and/or located at) piece of equipment 304. For example, some of the statements may be requests for different types of information. Any of the technologies listed above for transmitting energy may also be used for transmitting information, and any of the technologies discussed for transmitting information may be used for transmitting energy.

Communications receiver 312 receives communications from a module associated with (and/or located at) piece of equipment 304. The communications received by communications receiver 312 may be in response to communications from communications transmitter 310. The communications received by communications receiver 312 may contain information about piece of equipment 304. In an embodiment, when adding a new piece of equipment or when initially setting up system 100, system 100 may be configured to include any information desired. For example, the information may include a type, a model number, and a version number associated with piece of equipment 304 as will be discussed in conjunction with FIG. 3B.

Flange 314 is for attaching piece of equipment 304 to portion 302. Flange 314 is optional. Other methods of attaching piece of equipment 304 to portion 302 may be used. For example, equipment 304 may fit snugly into a slot or bin. Piece of equipment 304 may rest in a depression in a shelf of rack 210. Portion 302 may include places for inserting screws or other fasteners directly on wall 204.

Engagement structures 316, 318, and 320 may be any structure that assists with fastening or holding piece of equipment 304 in place. For example, a set of fasteners (not shown, e.g., screws, rivets, nuts, and/or bolts) may be associated with, or included in, engagement structure 316, 318, and 320. Engagement structures 316, 318, and 320 may be holes for accepting the fasteners. Although three engagement structures 316, 318, and 320 are shown, in other embodiments there may be any number of engagement structures on flange 314. For example, there may be only one engagement structure on flange 314. In another embodiment, there may be two, four, five, six, seven, eight, nine, ten, fifteen, twenty, or any other number of engagement structures on flange 314. In another embodiment, flange 314 and engagement structures 316, 318, and 320 are optional, because the piece of equipment may rest on one of shelves 214a-e or on a shelf within rack 210, for example. Flange 314, and engagement structures 316, 318, and 320 are discussed further in conjunction with engagement structures 334, 336, and 338.

Control panel 322 is optional. If present, control panel 322 may include any number of dials, meters, output devices, and/or input devices (e.g., a keypad, levers, buttons, and/or dials). Whether control panel 322 is present depends on, and the specifics of control panel 322 depend on, the specifics of piece of equipment 304.

EIM 324 is just one example of an EIM. EIM 324 communicates with EDM 306. EIM 324 provides EDM 306 information about piece of equipment 304. EIM 324 is discussed further in the course of discussing energy receiver 328, communications receiver 330, and communications transmitter 332.

Flange 326 connects with flange 314 for engaging piece of equipment 302 and holding piece of equipment 324 in place. Flange 326 is for attaching piece of equipment 304 to portion 302, via flange 314. Similar to flange 314, flange 326 is optional, and other methods of attaching equipment 304 to portion 302 may be used. Flange 326 is discussed further in the course of discussing engagement structure 334, 336, and 338.

Energy receiver 328 of EIM 324 receives energy from energy transmitter 308 of EDM 306. The energy received may be used to partly or completely power EIM 324. Energy receiver 328 may be any of a number of devices for receiving power. Energy receiver 328 may connect directly to energy source 308. Alternatively, energy receiver 328 may not be physically connected to energy source 308, but instead energy receiver may receive energy transmitted across a gap between energy source 308 and energy receiver 308. If energy source 308 is a light source, energy receiver 328 may be a photocell or other means of converting light into energy, for example. In other embodiments, energy receiver 328 may receive other forms and/or wavelengths of electromagnetic energy, such as microwaves, radio waves, and/or X-rays, for example. Some other examples of wavelengths of light that may be received by energy receiver 328 are ultraviolet light and infrared light.

In an embodiment, energy receiver 328 may receive monochromatic light, or a combination of wavelengths, depending on the characteristics of the energy receiver 328, for example. Energy receiver 328 may receive electrical energy via energy source 308. For example, energy source 308 may transfer electricity to energy receiver 328 upon contact. If energy source 308 transfers mechanical energy, energy receiver 328 may receive mechanical energy, such as various types of vibrations, which may be converted into electrical energy. Energy receiver 328 may include any combination of any of the various energy receivers listed above and/or any other energy receiver as long as energy receiver 328 is capable of receiving at least some of the energy transmitted from energy source 308. Energy receiver 328 may be a single energy receiver or may be a system including a combination of any number of energy receivers. Energy receiver 328 may include several energy receivers each compatible with a different type of EDM 306 having a different type of energy source 308.

Communications receiver 330 receives communications from energy transmitter 310 of EDM 306. Communications receiver 330 may receive queries from energy transmitter 310. Communications receiver 330 may only be capable of receiving a single type of signal, which is sent by communications transmitter 310 as a statement that requests all of the information available about piece of equipment 304. Alternatively, communications receiver 330 may be capable of receiving from communications transmitter 310 signals chosen from a set of many different signals in which each signal is a different statement or may make up a portion of statement. Each statement may cause EIM 324 to perform a different operation. For example, some of the statements may request that EIM 324 to provide different types of information to EDM 306.

Communications transmitter 332 transmits communications to communications receiver 312. The communications sent by communications transmitter 332 to communications receiver 312 may be in response to communications from communications transmitter 310. The communications sent by communications transmitter 332 may contain information about piece of equipment 304. In an embodiment, when adding a new piece of equipment or when initially setting up system 100, EIM 324 may be configured to include any information desired. The information that was transmitted by transmitter 332 is the information stored by EIM 324, which is discussed in conjunction with the machine-readable that stores that information in EIM 324.

Similar to engagement structure 316, 318, and 320, engagement structures 334, 336, and 338 may be any means that aids in holding piece of equipment 304 in a fixed place. For example, 334, 336, and 338 may include fasteners or may be holes for accepting the fasteners, as discussed in conjunction with engagement structure 316, 318 and 320. Similar to flange 314, although three engagement structures 334, 336, and 338 are shown, in other embodiments there may be any number of engagement structures on flange 326. For example, there may be only one engagement structure on flange 326. In another embodiment, there may be two, four, five, six, seven, eight, nine, ten, fifteen, twenty, or any other number of engagement structures on flange 326.

In the embodiment illustrated, the combination of flanges 314 and 326 and their respective engagement structures and flanges (e.g., engagement structure 316, 318, 320, 334, 336, and 338), and any additional fasteners (if necessary) need to be strong enough for supporting the weight of equipment 304. Additionally, in an embodiment, the combination of flanges 314 and 326 and their respective engagement structures and fasteners (if present) need to be adequate for holding equipment 304 in an essentially fixed position with respect to portion 302. Flange 326 and engagement structures 334, 336, and 338 are optional, because piece of equipment 304 may rest on a shelf, for example. If piece of equipment 304 rests on a shelf, the combination of flanges 314 and 326 and their respective engagement structures (e.g., engagement structure 316, 318, 320, 334, 336, and 338), and any fasteners only need to be strong enough to hold piece of equipment 304 in a fixed position. In other words, if piece of equipment 304 rests on a shelf, the combination of flanges 314 and 326 and their respective engagement structures (e.g., engagement structure 316, 318, 320, 334, 336, and 338), and any fasteners does not need to be strong enough to support piece of equipment 304.

In an embodiment, the combination of flanges 314 and 326, their respective engagement structures (e.g., engagement structures 316, 318, 320, 334, 336, and 338), and any fasteners keep piece of equipment 304 in a position that is only relatively fixed. Specifically, the position may need to be kept fixed to a degree that is adequate for keeping the module on the piece of equipment 304 in communication with EDM 306. For example, piece of equipment 304 may move or shift while fixed to portion 302 as long as the movement does not disrupt the communications between EDM 306 and the module associated with piece of equipment 304. Any interruptions sending energy or signals may need to be kept small enough so that the module associated with piece of equipment 304 is capable of responding to signals from EDM 306. In an embodiment, if EDM 306 receives indications that equipment 304 is present, but is not able to communicate with the module associated with piece of equipment 304, EDM 306 sends signals to a console indicating that piece of equipment 306 is present but is not communicating with EDM 306. Console 206 may then indicates to a user that equipment 304 is present but is not communicating, is noncompliant, and/or is improperly installed. In an embodiment, a determination is made as to whether piece of equipment 304 is improperly installed or is a non-compliant piece of equipment.

FIG. 3B shows an example of a security system 340 associated with an EIM 324 of FIG. 3A. FIG. 3B shows the back 342 of EIM 324 and a security tape 344. In other embodiments, security system 340 may include additional components and/or may not include all of the components listed above.

Security system 340 may deter and/or safeguard against a user improperly, illegally, and/or maliciously removing the piece of equipment 304. Specifically, security system 340 indicates when EIM 324 has been removed from piece of equipment 304. Security tape 344 is placed on the back 342 of EIM 324. Security tape 344 adheres to piece of equipment 304. When EIM 324 is removed from piece of equipment 304 security system 340 tends to break. In an embodiment, the breaking of security tape 344 breaks a circuit and therein causes an indication that EIM 324 has been removed from piece of equipment 304. In an embodiment, the removal of security tape 344 may be used as an indication that EIM 324 has been removed from piece of equipment 304 and reinstalled on another piece of equipment. In an embodiment, once security tape 344 is broken EIM 324 no longer functions. In an embodiment, the breaking of security tape 344 triggers an alarm. In an embodiment, when security tape 344 is broken, if EIM 324 is placed in a location where EIM 324 is still capable of communicating with EDM 306, a message is sent from EIM 324, via EDM 306, to one or more of consoles 106, 112, and/or 116. The purpose of the message may be to indicate that the security tape 344 is broken. In an embodiment, EIM 324 includes a circuit for storing an indication that security tape 344 was broken. Thus, in this embodiment, system 100 may be able to detect that EIM 324 was removed from a piece of equipment 304 and placed on another piece of equipment even if security tape 344 is repaired.

FIG. 3C shows an example of a circuit diagram of an EIM 327 of FIG. 3A. EIM 327 may include energy receiver 328, communications receiver 330, communications transmitter 332, contingency interface 333, power unit 346, and Central Processing Unit (CPU) 348. CPU 348 may include communications control 350, power management and distribution 352, central processing 354, and data interface 356. EIM 327 may also include non-volatile memory 358 and security tape 344. In other embodiments, EIM 327 may include additional components and/or may not include all of the components listed above.

The circuit diagram of EIM 327, in FIG. 3C, represents a circuit diagram of a different embodiment than the embodiment of EIM 324 of FIG. 3A. Specifically, the embodiment of EIM 327 of FIG. 3C includes contingency interface 333, and the embodiment of EIM 324 of FIG. 3A does not. However, EIM 327 is otherwise the same as EIM 324. In this specification, unless indicated otherwise, anywhere in which EIM 324 is mentioned EIM 327 may be substituted, and anywhere in which EIM 324 is mentioned EIM 327 may be substituted.

Energy receiver 328, communications receiver 330, and communications transmitter 332 form a part of a primary interface and are discussed above in conjunction with FIG. 3A. Alternative interface 333 is an alternative (or contingency) connection capable of performing the same functions as energy receiver 328, communications receiver 330, and communications transmitter 332. Alternative interface 333 is provided for situations in which the primary interface is not useable, inconvenient, or less convenient than alternative interface 333 for communicating with EDM 306. For example, if piece of equipment 304 is too close to wall 204, then there may not be room between wall 204 and piece of equipment 304 within which to place EIM 327 and EDM 306. Consequently, it may necessary to place EIM 327 and EDM 306 in locations in which they do not face one another preventing the use of the primary interface. In such a situation, alternative interface 333 may be used instead of the primary connection.

In an embodiment, alternative interface 333 is an electrical connection. In another embodiment, alternative interface 333 is an optical cable. In this embodiment if the primary interface uses optical communications via the atmosphere, then the primary difference between alternative interface 333 and the primary connection may be whether the light signals travel through the air or through a fiber optic cable.

Power unit 346 supplies energy to EIM 327 and may store energy supplied via EDM 306. Power unit 346 may include a power store, which may be any one of, any combination of, or all of a capacitor, a rechargeable battery, and/or a non-rechargeable battery, for example. By using a power store, EDM 306 may not need to supply power to EIM 327 at the same rate that EIM 327 consumes power when EIM 327 is active (e.g., when EIM 327 is processing a request from EDM 306 or supplying information to EDM 306). For example, EIM 327 may be capable of at least partially recharging while inactive so that while EIM 327 is active less power needs to be supplied. Alternatively, power unit 346 may be just a direct electrical connection between energy receiver 328 and CPU 348. If the power requirements of EIM 327 are small enough and the lifetime of the non-rechargeable battery is long enough, a non-rechargeable battery may be used without any additional power supplied by EDM 306, without a capacitor, and/or without a rechargeable battery. If power unit 346 includes a non-rechargeable battery, after the battery is dead the battery is replaced. Alternatively, after the battery is dead the entire EIM 327 is replaced.

CPU 348 may include hardware and/or software for managing power, storing information (such as while in a setup phase), retrieving information, processing and responding to signals from EDM 306, and/or for responding to security tape 344.

Communications control 350 controls communications sent by communications transmitter 332. Communications control 350 may also receive and at least partially process signals from communications receiver 330.

Power management and distribution 352 manages and distributes power from power unit 346. Power management and distribution may limit the amount of power transmitted to CPU 348 while EIM 327 is inactive and/or active. Power management and distribution 352 may also request power from EDM 327.

Central processing 354 may respond to breaks in security tape 344. Central processing 354 may coordinate the activities of communications control 350, power management and distribution 352, data interface 356, and security tape 344. Central processing may perform searches on memory 358 based on requests submitted via EDM 306.

Data interface 356 allows CPU 348 to interface with memory 358. Information is written to and retrieved from memory 358 via data interface 356.

Memory 358 may be long term memory, such as non-volatile memory. Memory 358 stores information about piece of equipment 304. The information may be stored in memory 358 may include parameters, limitations, capacities, requirement, and/or operations (e.g., tasks and functions) associated with the capabilities of piece of equipment 304. The information may include a complete description of the piece of equipment 304, any information that may be desired for characterizing better understanding of piece of equipment 304, and/or any information that is useful in distinguishing piece of equipment 304 from other pieces of equipment.

FIG. 3D shows an example of a circuit 360 that is associated with EDM 306 of FIG. 3A. Circuit 360 includes power source 362 and concentrator 364. Concentrator 364 includes power management and distribution 366, EDM communications 368, central processing 370, non-volatile memory 372, console communications 374, communications connection 376, power connections 378, and EDMs 380a-n. In other embodiments, circuit 360 may include additional components and/or may not include all of the components listed above.

Power source 362 powers circuit 360. Some of the power from power source 362 may be sent to the EIMs (such as EIMs 324, and/or 327) that correspond to EDMs 380a-n.

Concentrator 364 controls the power flow to the EIMs (such as EIMs 324, and/or 327). Concentrator 364 also controls communication signals sent by EDMs 380a-n to the EIMs, the handling of communications received by EDMs 380a-n from the EIMs, and communications between EDMs 380a-n and the consoles 106,112, and/or 116. Concentrator 364 may be software and/or hardware. Concentrator 364 may be incorporated within the hardware and/or software of consoles 106, 112, and/or 116. Alternatively, concentrator 364 may be a separate unit than consoles 106, 112, and/or 116 or may be a set of functions that are distributed among EDMs 380a-n.

Power management and distribution 366 manages and distributes power from power source 362 to EDMs 380a-n and the corresponding EIMs (such as EIMs 324, and/or 327). In an embodiment, power management and distribution 366 essentially ensures that each of the EIMs have sufficient power to supply information when requested.

EDM communications 368 control communications sent and received by EDMs 380a-n. EDM communications 368 causes EDMs 380a-n to send requests for information and receive information based on requests for information sent by consoles 106, 112, and/or 116.

Central processing 370 may coordinate the activities of EDM communications 368 and power management and distribution 368. Central processing may perform searches for information requested by consoles 106, 112, and/or 116.

Non-volatile memory 372 stores information retrieved from the EIMs. Non-volatile memory 372 may include a database and/or an application that causes concentrator 364 to automatically collect information from the EIMs (such as EIMs 324, and/or 327) and EDMs 380a-n. Non-volatile memory 372 stores the positions of the EDMs, from which the position of proximate EIMs, and thus a piece of equipment can be inferred.

Console communications 374 interfaces with consoles 106, 112, and/or 116. Console communications 374 receives requests from consoles 106, 112, and/or 116 for information about piece of equipment 304 and/or a location for storing piece of equipment 304 and sends information about piece of equipment 304 and the storage locations to consoles 106, 112, and/or 116.

Communications connection 376 connects EDM communications 368 to EDMs 380a-n. Communications connection 376 may be used for sending communications between EDM communications 368 and EDMs 380a-n. Communications connection 376 may include separate wires for each communications transmitter and receiver. Alternatively, communications connection 376 may be a multi-access channel. Communication connection 376 may be a physical connection. Alternatively, communication connection may not be a physical connection, and may be a radio wave or optical connection, for example.

Power connection 378 connects power management and distribution 366 to the power transmitters of EDMs 380a-n. Power connection 378 is used for sending power to EDMs 380a-n based upon the power management and distribution 366.

EDMs 380a-n may be the same as EDMs 202a-g, discussed in connection with FIG. 2. EDMs 380a-n may be the EDMs associated with a single rack, a single row, or a group of slots. EDMs 380a-n may be the EDMs associated with the entire system 100.

FIG. 3E shows a circuit diagram of EDM 382. EDM 382 includes energy source 308, transmitter 310, receiver 312, receiver lead 384, transmitter lead 386, source lead 388, return lead 390, primary interface 394, contingency interface 392, and contingency connector 393. In other embodiments, EDM 382 may include additional components and/or may not include all of the components listed above.

The circuit diagram of EDM 382, in FIG. 3E, represents a circuit diagram of a different embodiment than the embodiment of EDM 306 of FIG. 3A. Specifically, the embodiment of EDM 382 of FIG. 3E includes contingency interface 393, and the embodiment of EDM 306 of FIG. 3A does not, but is otherwise the same.

Receiver lead 384 is used by receiver 312 for receiving signals. Transmitter lead 386 is used by transmitter 310 for transmitting signals. Source lead 388 is used by energy source 308 for transmitting energy to an EIM (such as EIM 324 and/or 327). Return lead 390 may be a return to ground or a return to the power source that powers EDM 382 to complete a circuit. Receiver lead 384 and transmitter lead 382 may be part of communication connection 376. Source lead 388 may be part of power connection 378. Return lead may also be part of power connection 378. Alternatively, each of communication connection 376 and power connection 378 may have their own return lead.

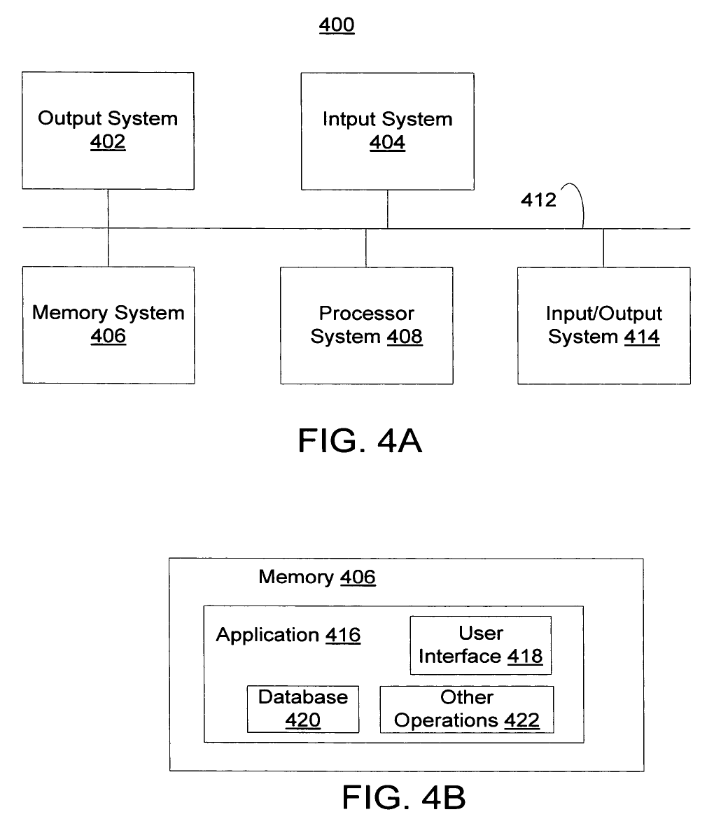

FIG. 4A shows a block diagram of a console 400 used in system 100. Console 400 may include output system 402, input system 404, memory system 406, processor system 408, communications system 412, and input/output device 414. In other embodiments, console 400 may include additional components and/or may not include all of the components listed above.

Console 400 is an example of a console that may be used for any one of, any combination of, or all of consoles 106, 112, and/or 114.

Output system 402 may include any one of, some of, any combination of, or all of a monitor system, a handheld display system, a printer system, a speaker system, a connection or interface system to a sound system, an interface system to peripheral devices and/or a connection and/or interface system to a computer system, an intranet, and/or an internet, for example.

Input system 404 may include any one of, some of, any combination of, or all of a keyboard system, a mouse system, a track ball system, a track pad system, buttons on a handheld system, a scanner system, a microphone system, a connection to a sound system, and/or a connection and/or interface system to a computer system, intranet, and/or internet (e.g., IrDA, USB), for example.

Memory system 406 may include, for example, any one of, some of, any combination of, or all of a long term storage system, such as a hard drive; a short term storage system, such as random access memory; a removable storage system, such as a floppy drive or a removable drive; and/or flash memory. Memory system 406 may include one or more machine-readable mediums that may store a variety of different types of information.

The term machine-readable medium is used to refer to any medium capable carrying information that is readable by a machine. One example of a machine-readable medium is a computer-readable medium. Another example of a machine-readable medium is paper having holes that are detected that trigger different mechanical, electrical, and/or logic responses. The term machine-readable medium also includes mediums that carry information while the information is in transit from one location to another, such as copper wire and/or optical fiber.

Processor system 408 may include any one of, some of, any combination of, or all of multiple parallel processors, a single processor, a system of processors having one or more central processors and/or one or more specialized processors dedicated to specific tasks.

Communications system 412 communicatively links output system 402, input system 404, memory system 406, processor system 408, and/or input/output system 414 to each other. Communications system 412 may include machine readable media such as any one of, some of, any combination of, or all of electrical cables, fiber optic cables, and/or means of sending signals through air (e.g., wireless communications), for example. Some examples of means of sending signals through air include systems for transmitting electromagnetic waves such as infrared and/or radio waves and/or systems for sending sound waves.

Input/output system 414 may include devices that have the dual function as input and output devices. For example, input/output system 414 may include one or more touch sensitive screens, which display an image and therefore are an output device and accept input when the screens are pressed by a finger or stylus, for example. The touch sensitive screens may be sensitive to heat and/or pressure. One or more of the input/output devices may be sensitive to a voltage or current produced by a stylus, for example. Input/output system 414 is optional, and may be used in addition to or in place of output system 402 and/or input device 404.

FIG. 4B shows memory system 406. Memory system 406 stores application 416, which includes user interface 418, database 420, and other functions 422. In other embodiments, memory system 406 may include additional components and/or may not include all of the components listed above. For example, memory system 406 may store other applications.

Application 416 includes one or more instructions that cause console 400 to implement a method of for retrieving information from the EIMs (such as EIM 324 and/or 327) via the EDMs (e.g., EDMs 202a-g, 212a-j, 306, 380a-n, and/or 382). Application 416 may include a database for storing information about the pieces of equipment stored in system 100. Application 416 may be part of another application or may not actually be a single application, but include a multiplicity of applications.

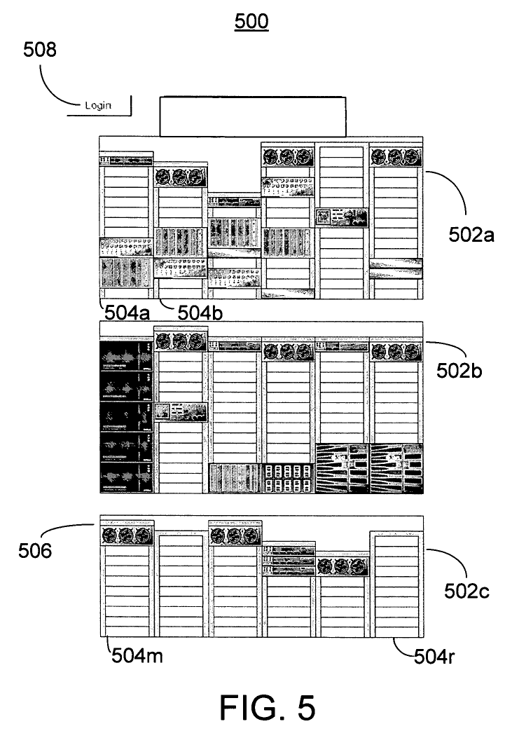

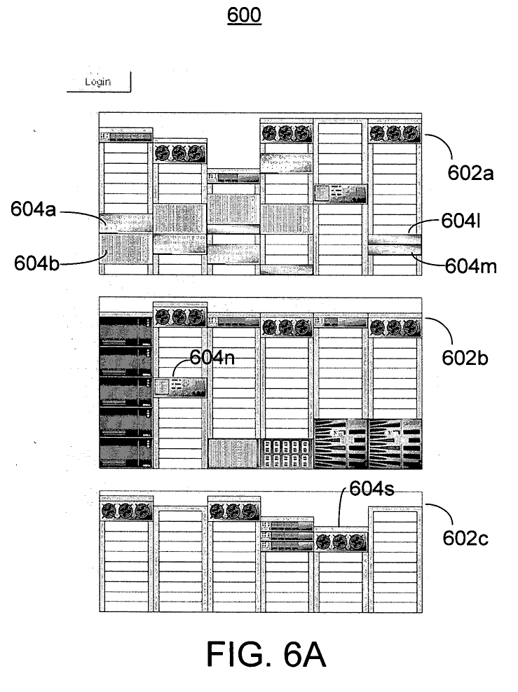

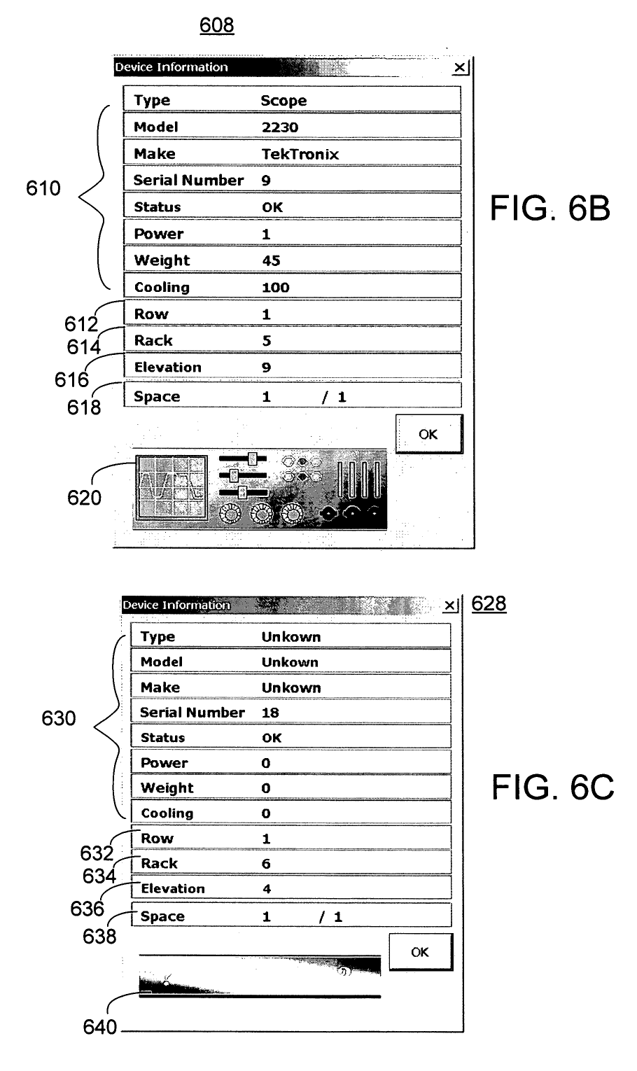

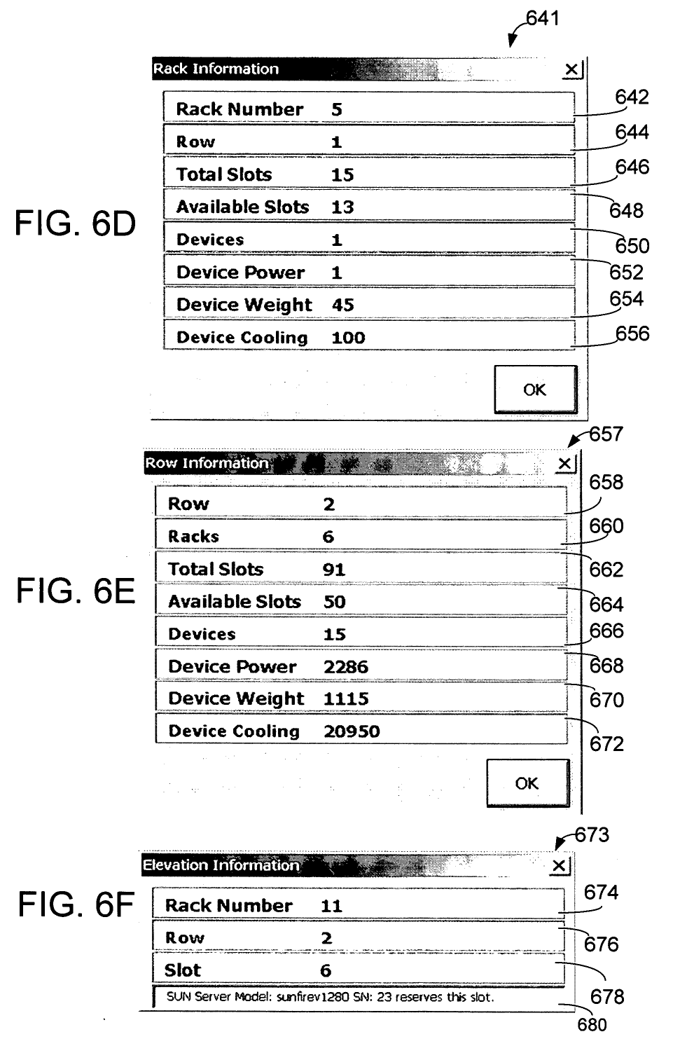

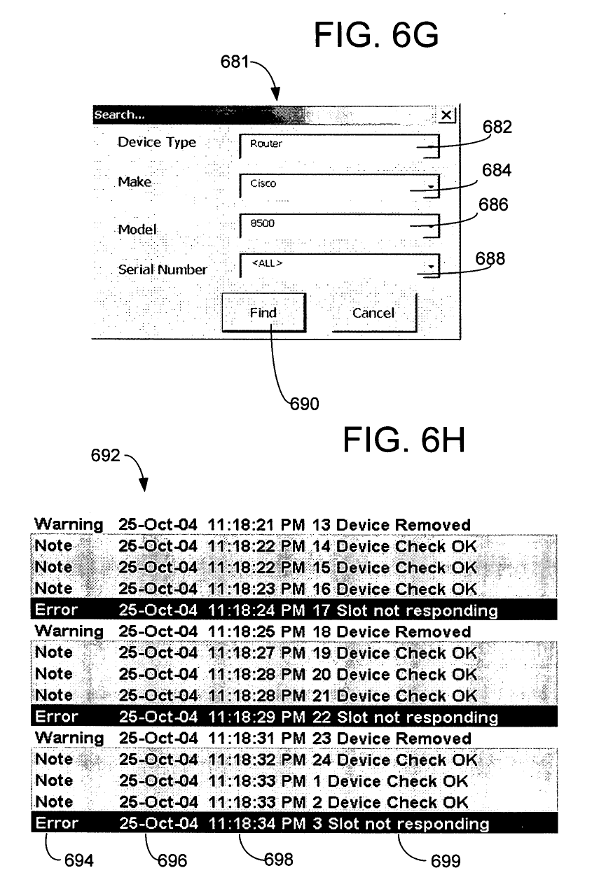

User interface 418 may be a Graphical User Interface (GUI) that the user may use for entering data, or viewing information collected, via the EDMs (e.g., EDMs 202a-g, 212a-j, 306, 380a-n, and/or 382) from the EIMs (such as EIM 324 and/or 327). User interface 418 may cause the EDMs to collect information from the EIMs, view, format, and/or search the database. User interface 418 may be Windows based, UNIX based, or based on any other, commercially available operating system. User interface 418 may include prompts and/or dialog boxes, for example. Examples of screenshots from an embodiment of user interface 418 appear in FIGS. 5-6H.

Database 420 stores the information collected from the EIMs (such as EIM 324 and/or 327) and EDMs (e.g., EDMs 202a-g, 212a-j, 306, 380a-n, and/or 382). Database 420 may be queried and searched, via numerous different methods, for the information stored. For example, in an embodiment, database 420 may include several different fields, which may each be searched individually. In another embodiment, database 420 may be searched, via keywords, across several fields in addition to or instead of searching the fields individually.

Other functions 422 may include software for controlling the EDMs. Other functions 422 may include software (e.g., a database server) for searching, storing, and/or otherwise accessing database 420. Other functions 422 may also include an interface for interfacing with other networks and/or consoles. Other functions 422 may include all of or part of concentrator 364. Alternatively, concentrator 364 may be a separate unit from other functions 422.

FIG. 5 is an example of a screenshot 500 of a Graphical User Interface (GUI) associated with system 100, such as user interface 418. Screen shot 500 includes one or more images of rows of racks 502a-c, including images of racks 504a-r, piece of equipment 506, and login button 508. In other embodiments, screenshot 500 may include additional components and/or may not include all of the components listed above.

Screenshot 500 may be available on any of consoles 106, 112, and/or 116. Screenshot 500 shows rows of racks 502a-c, which are part of system 100. For example, rows of racks 502a-c may represent racks 104a-c and/or 110a-c, which are associated with local facilities 102 and 108, respectively. The location of pieces of equipment in racks 504a-r, such as piece of equipment 506, may be identified by row (e.g., 502c), rack (e.g., 504m), and position within the rack. Piece of equipment 506 may represent piece of equipment 304 or another piece of equipment, for example. In an embodiment, each piece of equipment is represented by an icon that is suggestive of its type. For example, an oscilloscope may include a display having a grid and a trace of a waveform on the display. The icon representing a particular piece of equipment (e.g., piece of equipment 506) may be stored in the EIM associated with that piece of equipment. Upon installing the piece of equipment, the icon may be downloaded to consoles 106, 112, and/or 114. In another embodiment, each piece of equipment is represented by an actual photograph of that piece of equipment.

Login button 508 is optional, and may be used for logging into system 100. Upon activating login button 508, a prompt appears requesting login information. Any of a number of other screenshots may be used for logging in. For example, the screenshot prompting a user to login may appear prior to displaying any of racks 502a-c. In an embodiment, pieces of equipment that are not identified may appear as empty positions within racks 502a-c. In another embodiment, pieces of equipment that were not identifiable may be represented by an icon representing a generic piece of equipment or may be represented by an icon that suggests that information is missing.

FIG. 6A is an example of a screenshot 600 associated with system 100. Screen shot 600 also includes one or more images of rows of racks 602a-c, including images of pieces of equipment 604a-s and login button 606. In other embodiments, screenshot 600 may include additional components and/or may not include all of the components listed above.

In contrast to screen shot 500, screen shot 600 includes icons for representing pieces of equipment that were not identifiable. Racks 602a-c could be the same as racks 502a-c, which are discussed above in conjunction with FIG. 5. Pieces of equipment 604a-s are the pieces of equipment stored within racks 602a-c. The icons used for pieces of equipment 604l and 604m are examples of icons that may be used to identify pieces of equipment that were not identifiable. The icons used for pieces of equipment 604l and 604m suggest information is missing. Other icons could be used that suggest missing information, such as a blank box or a single question mark. In FIG. 6A, pieces of equipment 604m and 604n are selected. In an embodiment, the selection of a piece of equipment is indicated by darkening the icon and/or changing the color of the icon. However, any of a variety of other methods may be used for indicating that the piece of equipment is selected. In an embodiment, upon selecting a piece of equipment a window appears showing information about the piece of equipment.

FIG. 6B is an example of an information window 608 that displays information about a selected piece of equipment of FIG. 6A. Window 608 includes equipment information fields 610, row field 612, rack field 614, elevation field 616, space field 618, and icon 620. In other embodiments, window 608 may include additional components (e.g., fields) and/or may not include all of the components listed above.

Window 608 is the window displayed in response to selecting piece of equipment 604n of FIG. 6A. In an embodiment, at least part of the information displayed in window 608 is information collected from one or more EIMs (such as EIMs 324 or 327). In the example illustrated, information fields 610 include the type of equipment, the model, make, serial number, the status, the power required, the weight, and the energy required for cooling piece of equipment 604n. In other embodiments, other information fields may be displayed. In an embodiment, different information fields are displayed for different types of equipment. In an embodiment, which information fields are displayed is determined by information in the EIM (such as EIM 324 or 327) supplying the information. Row field 612 and rack field 614 identify the row and rack, respectively, at which piece of equipment 604n is located. Elevation field 616 identifies how high in the rack piece of equipment 604n is located (e.g., in which slot piece of equipment 604n is located). Space field 618 indicates the amount of space (e.g., how many slots) piece of equipment 604n requires. Icon 620 is optional. Icon 620 depicts the icon used in screenshot 600 to represent piece of equipment 604n.

FIG. 6C is an example of an information window 628 that displays information about a selected piece of equipment of FIG. 6A in which the piece of equipment was not identifiable. Window 628 includes equipment information fields 630, row field 632, rack field 634, elevation field 636, space field 638, and icon 640. In other embodiments, window 628 may include additional components (e.g., fields) and/or may not include all of the components listed above.

Window 628 is the window displayed in response to the selection of piece of equipment 604m of FIG. 6A. In the example illustrated, similar to FIG. 6A, equipment information fields 630 include the type of equipment, the model, make, serial number, the status, the power required, the weight, and the energy required for cooling piece of equipment 604m. However, since piece of equipment 604m was not identifiable the values for the type of equipment, the model, and the make are values such as “unknown,” which indicate a lack of information. Similarly, in this example, piece of equipment 604m is not operationally installed within its rack. Consequently, values of “0” appear in the fields for the power and the energy required for cooling piece of equipment 604m. In this embodiment a “0” also appears for the unknown value of the weight. However, in other embodiments, the value “unknown” may be used. In other embodiments, other information fields may be displayed. The information for row field 632, rack field 634, and elevation field 636 are provided by the EDM that discovered the EIM associated with piece of equipment 604m, and identify the row, rack, and elevation, respectively, at which piece of equipment 604m is located. Elevation field 636 may identify how high in the rack piece of equipment 604n is located (e.g., in which slot equipment 604n is located). Space field 638 indicates the amount of space (e.g., how many slots) piece of equipment 604n requires. Icon 640 is optional. Icon 640 depicts the icon used in screenshot 600 to represent an unknown piece of equipment, and therefore icon 640 is used to represent piece of equipment 604m.

FIG. 6D is an information window 641 for a rack. Window 641 includes a rack field 642, rows in rack field 644, total rack positions field 646, available rack positions field 648, rack devices field 650, rack device power field 652, rack device weight field 654, and rack device cooling field 656. In other embodiments, window 641 may include additional components (e.g., fields) and/or may not include all of the components listed above.

Window 641 includes information about a specific rack. In an embodiment, window 641 appears in response to selecting a rack displayed on a screen.

Rack field 642 includes an identification of the rack represented by window 641. The identification may be a number corresponding to a rack within a row. Alternatively, the identification in rack field 642 may be an alphanumeric code or a name (identifying the rack) that may be chosen in a manner that is not related to the position of the rack within the row. Rows in rack field 644 contains a value that represents the number of rows in the rack corresponding to window 641. Total rack positions field 646 contains a value that represents the total number of positions within the rack in which equipment can be placed. Available rack positions field 648 contains a value that represents the number of positions that are empty and therefore available for placing new pieces of equipment. Rack devices field 650 contains a value that represents the total number of devices (e.g., pieces of equipment) in the rack. Rack device power field 652 contains a value that represents the total power consumed by the devices in the rack. Rack device weight field 654 contains a value that represents the sum of the weights of all of the devices in the rack. Rack device cooling field 656 contains a value representing the total power necessary to cool all of the devices in the rack.

FIG. 6E is an information window 657 for a row. Window 657 includes a row field 658, racks in row field 660, total row positions field 662, available row positions field 664, row devices field 666, row device power field 668, row device weight field 670, and row device cooling field 672. In other embodiments, window 657 may include additional components (e.g., fields) and/or may not include all of the components listed above.

Row field 658 includes an identification of the row represented by window 657. The identification may be a number corresponding to a position within a row. Alternatively, the identification may be an alphanumeric code or a name (identifying the row) that may not be chosen in a manner that is related to the position of the row within the local facility 102 and/or 108. Racks in row field 660 contains a value that represents the number of racks in the row. Total slot positions field 662 contains a value that represents the total number of positions within the row in which pieces of equipment can be placed. Available slots positions field 664 contains a value that represents the number of positions within the row that are empty and therefore available for placing new equipment. Row devices field 666 contains a value that represents the total number of devices (e.g., pieces of equipment) in the row. Row device power field 668 contains a value that represents the total power consumed by all discovered devices in the row. Row device weight field 670 contains a value that represents the sum of the weights of all discovered devices in the row. Row device cooling field 672 contains a value representing the total power necessary to cool all of the devices in the row.

FIG. 6F shows an information window 673 for a position in a rack. Window 673 includes rack number field 674, row number field 676, position number field 678, and device information field 680. In other embodiments, window 673 may include additional components and/or may not include all of the components listed above.

Window 673 appears in response to selecting a particular position. In an embodiment, window 673 appears whether or not a piece of equipment occupies the position. Rack number field 674 contains a value that represents the rack containing the position selected. Row number field 676 contains a value that represents the row containing the position selected. Position number field 678 contains a value that represents the position selected within the rack. Position information field 680 contains information related to the usage of the position. If the position is empty and available for use, position information field 680 may contain a value of “empty” or “available.” If the position is empty, because the piece of equipment associated with the position was temporarily removed, position information field 680 may state that the position is reserved for the missing piece of equipment. Window 673 may be used to determine whether there is a conflict in the intended usage of a particular position. Window 673 may indicate whether a position is actually occupied by a piece of equipment. Window 673 may indicate that a position needs to be kept empty. For example, a position may need to be kept unoccupied so that a piece of equipment above and/or below the position can be properly cooled or accessed.

FIG. 6G shows a search window 681 of system 100. Search window 681 includes a device type field 682, make field 684, model field 686, serial number field 688, and find button 690. In other embodiments, window 681 may include additional components (e.g., search fields) and/or may not include all of the components listed above.

Search window 681 may be used for finding information about one or more pieces of equipment. Pieces of equipment may be found by searching for (1) a particular type of piece or range of pieces of equipment using type field 682, (2) a particular make of the piece of equipment or a particular range of pieces of equipment using make field 684, (3) a particular model or range of models using model field 686, and/or (4) a particular serial number or range of serial numbers using serial number field 688. In an embodiment, search window 681 may accept wildcards for characters in any one of, any combination of, and/or all of the fields of widow 681. In an embodiment, search window 681 may have an additional field in which all fields may be searched simultaneously. Find button 690 may be used for causing a search to begin. After a set of one or more search terms are entered into the fields of search window 681, find button 690 may be selected to start the search.

FIG. 6H shows a log 692 to monitor changes in system 100 as the changes occur. Log 692 includes severity column 694, a date column 696, a time column 698, and a description column 699. In other embodiments, log 692 may include additional components (e.g., columns) and/or may not include all of the components listed above.

Log 692 records changes to system 100 as the changes happen. In an embodiment, when a piece of equipment is removed, returned, and/or stops working, an entry is automatically added to log 692. Severity column 694 includes an indication, such as “note,” “error,” or “warning,” that indicates the severity and/or nature of the entry. Date column 696 indicates the day at which the change occurred. Time column 698 indicates the time at which the change occurred. Description column 699 includes a description of the event. In an alternative embodiment, the information in log 692 may be organized in another manner. For example, log 692 may be organized according to rows (e.g., having a severity row, a date row, time row, and description row) instead of being organized by column. In other embodiments, other information may be included in log 692 in addition to or instead the columns discussed above.

In an embodiment, EDMs (such as EDMs 202a-g, 212a-j, 306, 380a-n, and/or 382), EIMs (such as EIM 324 and/or 327), and software (e.g., application 416) are included in a kit, which is sold separately from the rest of system 100. In another embodiment, the kit may also include any part of or all of circuit 360. The kit may be installed in a system to convert the system into one such as system 100.

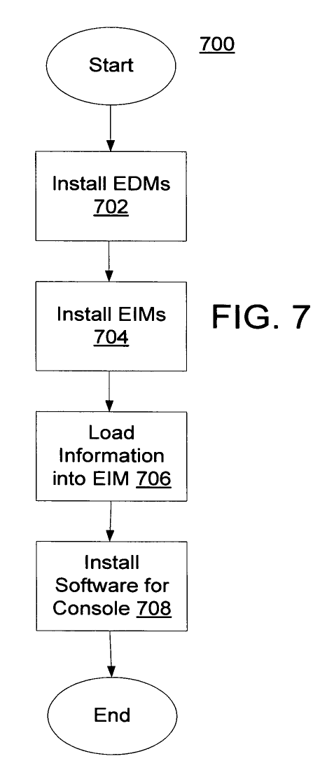

FIG. 7 shows a method of converting a system into one such as system 100 using such a kit. In step 702, the EDMs (e.g., EDMs 202a-g, 212a-j, 306, 380a-n, and/or 382) are installed on the racks (e.g., racks 200 and/or 210) and communicatively coupled to a console such as one of consoles 106, 112, and/or 116. In step 704, EIMs (such as EIM 324 and/or 327) are installed on the pieces of equipment associated with system 100. The EIMs are positioned within racks of system 100 and the EDMs are positioned on the pieces of equipment such that when the pieces of equipment are installed the EDMs and EIMs are communicatively coupled. In step 706, information about the pieces or equipment is loaded onto the EIMs. The information may be loaded onto the EIMs, and the EIMs may be affixed to the pieces of equipment at the location where the pieces of equipment are manufactured, at the site of system 100, or anywhere else. In step 708, the software (e.g., application 416) for interfacing with the EDMs and for providing the GUI (e.g., the screenshots associated with FIG. 6A-H) is installed on consoles 106, 112, and/or 116.

The steps of method 700 may be performed simultaneously and/or in any order with respect to one another. In other embodiments, method 700 may include additional steps and/or may not include all of the steps listed above.

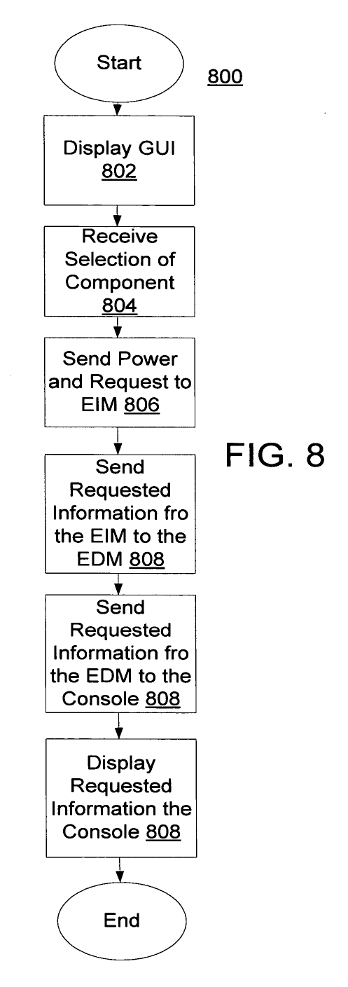

FIG. 8 shows an example of a manner in which system 100 may operate. In step 802, a GUI (which may be an embodiment of user interface 418) is presented to one or more users at one or more of consoles 106, 112, and 116. The console may query the EDMs, via concentrator 364, for location information and device information so that the GUI may display an image showing pieces of equipment and the location of the pieces of equipment within the racks associated with system 100. For example, one of consoles 106, 112 or 116 may query one or more of EDMs 202a-g, 212a-j, 306, 380a-n, and/or 382 for location information and device information. The results of the query may be used by the GUI to display an image showing pieces of equipment and the location of the pieces of equipment within racks 104a-c or 110a-c. The EDMs, under the control of concentrator 364, may query the EIMs (such as EIM 324 and/or 327) for the equipment information necessary to construct the screenshots including one or more of the user interfaces associated with screenshots of FIGS. 6A-H. In step 804, the user using the GUI selects a piece of equipment. The console receives the selection of the piece of equipment, and sends a signal via concentrator 364 to an EDM associated with the selected piece of equipment. The EDM, under the control of concentrator 364, powers the EIM and just prior to, and/or while, sending a signal to and/or receiving a signal from the EIM requesting information. Alternatively, the EIM may be continually powered by the EDM as determined by concentrator 364. Alternatively, the EIM may request power, when needed, from the EDM. In step 806, the EIM receives the power and the request. In step 808, EIM responds by sending the information requested to the EDM. In step 810, the EDM sends, via concentrator 364, the information received to one or more of the consoles 106, 112, and 116. In step 812, the one or more consoles receiving the information displays a window having the information received on a display for the user. In other embodiments, method 800 may include additional steps and/or may not include all of the steps listed above.

Although the invention has been described with reference to specific embodiments, it will be understood by those skilled in the art that various changes may be made and equivalents may be substituted for elements thereof without departing from the true spirit and scope of the invention. In addition, modifications may be made without departing from the essential teachings of the invention.

1. A system comprising:
at least one storage location;
at least one module that stores and transmits information about an item of interest; and
at least one module that requests and receives information about the item of interest, the module that requests and receives being
(a) associated with the storage location, and
(b) capable of reading the information from the module that stores and transmits.

2. The system of claim 1, wherein
(I) the item of interest is a piece of equipment;
(II) the storage location includes at least one or more rows of racks, wherein
(A) each row of racks includes at least one or more racks, and
(B) each rack includes one or more slots;
(III) the at least one module that stores and transmits includes at least
(A) a first light source for transmitting signals,
(B) a first photocell for receiving signals,
(C) a photocell for receiving power,
(D) a first electrical connector for
(a) transmitting signals,
(b) receiving signals, and
(c) receiving power, and
(d) the first electrical connector being in parallel with the first light source, the first photocell for receiving signals and the photocell for receiving power,
(E) a power unit capable of
(a) receiving power in the form of electricity, via the first electrical connector, and
(b) receiving power in the form of electricity from the photocell for receiving power,
(F) a security strip,
(G) nonvolatile memory,
(H) a processor that is powered by the power unit, the processor being capable of at least
(a) receiving a signal from the security strip indicating a breach in security,
(b) managing and distributing power for the power unit,
(c) receiving signals from the first photocell for receiving signals,
(d) receiving signals from the first electrical connector,
(e) storing information in the nonvolatile memory,
(f) accessing information in the nonvolatile memory
(g) causing the first light source for transmitting signals to transmit a signal including the information accessed, and
(h) causing the first electrical connector to transmit a signal including the information accessed;
(IV) the at least one module that requests and receives including at least
(A) a second light source for transmitting signals to the first photocell for receiving signals,
(B) a second photocell for receiving signals from the first photocell for transmitting signals,
(C) a photocell for transmitting power to the photocell for receiving power,
(D) a second electrical connector for
(a) transmitting signals to the first electrical connector,
(b) receiving signals from the first electrical connector, and
(c) transmitting power to the first electrical connector,
(d) the second electrical connector being in parallel with at least
(1) the second light source for transmitting signals,
(2) the second photocell for receiving signals, and
(3) the photocell for transmitting power, and
(E) a concentrator for at least
(a) controlling the second light source for transmitting signals,
(b) controlling the light source for transmitting power, and
(c) controlling the second photocell for receiving power; and
(V) the system further comprising at least one console configured for at least
(A) communicating with the concentrator,
(B) maintaining a database based, at least in part, on the information accessed, and
(C) capable of displaying a graphical user interface, the graphical user interface displaying at least information related to
(a) characteristics of the piece of equipment associated with the information accessed,
(b) a location associated with the piece of equipment,
(c) an availability of the one or more slots, and
(d) a log that monitors changes to the system related to the availability of the one or more slots, in which the log is updated essentially as the changes occur.

3. A system comprising: a module that stores information about an item of interest and is capable of being coupled to the item of interest, the module including at least
a transmitter for transmitting information about the item of interest; and
a receiver of energy that receives energy from an external power source, the energy being used to power the operations of the module.

4. The system of claim 3, wherein the signal requesting information requests a subset of a set of information stored at the item of interest.

5. The system of claim 3, wherein the transmitter transmits light.

6. The system of claim 3, wherein the receiver of energy receives light.

7. The system of claim 3, wherein the receiver of energy is a photocell.

8. The system of claim 3, wherein the receiver of energy is a connector for making an electrical connection.

9. The system of claim 3, wherein the module also includes at least a processor for controlling the transmitter.

10. The system of claim 3, wherein the module also includes at least a machine-readable medium for storing the information.

11. The system of claim 3, wherein the module also includes at least a receiver that is capable of receiving requests for the information.

12. A system comprising: a module that receives information about an item of interest and is capable of communicating with an information module located at the item of interest, the module that receives includes at least
a receiver for receiving information about the item of interest; and
a transmitter of energy that transmits energy to the information module to power operations of the information module.

13. The system of claim 12, wherein the receiver receives light.

14. The system of claim 12, wherein the transmitter of energy transmits light.

15. The system of claim 12, wherein the transmitter of energy is a light emitting diode.

16. The system of claim 12, wherein the transmitter of energy is a connector for making an electrical connection.

17. The system of claim 12, wherein the transmitter of energy transmits light, and the module also includes at least another transmitter of energy that transmits electrical energy.

18. The system of claim 12, wherein the module also includes at least a transmitter that transmits requests for information.

19. The system of claim 18, wherein the transmitter that transmits requests also transmits light.

20. The system of claim 18, wherein the transmitter that transmits requests includes at least a light emitting diode.

21. The system of claim 12, wherein the module that receives is coupled to a module for managing a power distribution among a plurality of modules that receive information.

22. The system of claim 12, wherein the module that receives is coupled to a concentrator that
manages a power distribution among a plurality of modules that receive information;
manages communication messages sent by the plurality of modules that receive information; and
manages communications with a console, wherein the console is capable of displaying a user interface.
